(19) United States
(12) Patent Application Publication
(10) Pub. No.: US 2017/0319077 A1
(43) Pub. Date: Nov. 9, 2017

(54) SAMPLE INFORMATION ACQUISITION APPARATUS

(71) Applicant: CANON KABUSHIKI KAISHA, Tokyo (JP)

(72) Inventors: Daisuke Nagao, Kawaguchi-shi (JP); Koichi Suzuki, Kodaira-shi (JP)

(21) Appl. No.: 15/526,890

(22) PCT Filed: Oct. 20, 2015
(86) PCT No.: PCT/JP2015/005286

§ 371 Date: May 15, 2017

(30) Foreign Application Priority Data
Dec. 9, 2014 (JP) 2014-249436

(51) Int. Cl. A61B 5/00; G01N 29/06; G01N 29/24

(52) U.S. Cl. CPC A61B 5/0095; A61B 5/742; A61B 5/7475; A61B 5/4312; G01N 29/2418; G01N 2291/02466; G01N 29/0654

(57) ABSTRACT

The present invention relates to a sample information acquisition apparatus including a determination unit that determines a state of contact between a probe and a sample and whether the sample is on an optical path on the basis of an ultrasonic echo signal of ultrasonic waves received by the probe for the ultrasonic waves transmitted from the probe, prior to generation of a photoacoustic image; and a control unit that causes a light irradiating unit to irradiate the sample with light on the basis of a result of the determination. The determination unit determines the state of contact on the basis of information about multiple ultrasonic echo signals received by multiple transducers that are set apart from each other, among the multiple ultrasonic echo signals received by the multiple transducers in response to the ultrasonic waves transmitted from the multiple transducers in the probe.

### TECHNICAL FIELD

The present invention relates to a sample information acquisition apparatus.

### BACKGROUND ART

Sample information acquisition apparatuses, such as photoacoustic image generation apparatuses, have hitherto been studied in medical fields. The photoacoustic image generation apparatuses irradiate samples with pulsed light and receive photoacoustic waves generated from the inside of the samples with probes to form images of shapes and functions of the inside of the samples.

In order to generate the photoacoustic waves from the inside of the samples and accurately acquire the photoacoustic waves with the probes in such a photoacoustic image generation apparatus, it is necessary to establish the correct positional relationship among the sample, the optical path, and the probe correct. In other words, it is preferred that the sample be on the optical path and that the sample be in close contact with the probe. This is because, if the sample is out of the optical path, only weak photoacoustic waves may be generated or no photoacoustic wave may be generated. In addition, if the sample is not in close contact with the probe, the photoacoustic waves may be reflected between the probe and the sample and may not reach the probe.

Accordingly, a photoacoustic image generation apparatus including a sensor that detects the state of contact between a sample and a probe is proposed (PTL 1) as a method of understanding the positional relationship between the sample, the optical path, and the probe.

### CITATION LIST

### Patent Literature

PTL 1: Japanese Patent Laid-Open No. 2012-187389

### SUMMARY OF INVENTION

### Technical Problem

In order to resolve the above problems, the state of contact between the sample and the probe is detected with the sensor in the photoacoustic image generation apparatus in related art. However, only the state of contact at a portion where the contact sensor is provided is detected with the above method. It is necessary to make improvements in, for example, reduction in time required to determine whether the sample is on the optical path and to determine whether the sample is in contact with the probe.

### Solution to Problem

The present invention provides a sample information acquisition apparatus including a light source; a unit of irradiating a sample with light from the light source; a probe including multiple transducers that transmit and receive ultrasonic waves to the sample; an image generation unit configured to at least generate a photoacoustic image on the basis of a photoacoustic signal of the ultrasonic waves received by the probe for the light radiated from the light irradiating unit to the sample; a determination unit configured to determine a state of contact between the probe and the sample and whether the sample is on an optical path on the basis of an ultrasonic echo signal of the ultrasonic waves received by the probe for the ultrasonic waves transmitted from the probe, prior to the generation of the photoacoustic image; and a control unit configured to cause the light irradiating unit to irradiate the sample with the light on the basis of a result of the determination in the determination unit. The determination unit determines the state of contact between the probe and the sample on the basis of information about multiple ultrasonic echo signals received by multiple transducers that are set apart from each other, among the multiple ultrasonic echo signals received by the multiple transducers in response to the ultrasonic waves transmitted from the multiple transducers in the probe.

According to the present invention, there is provided a sample information acquisition apparatus capable of determining whether the sample is on the optical path and the state of contact between the sample and the probe with a high accuracy in a short time.

Further features of the present invention will become apparent from the following description of exemplary embodiments with reference to the attached drawings.

### BRIEF DESCRIPTION OF DRAWINGS

FIG. 1 is a block diagram of a sample information acquisition apparatus in a first embodiment of the present invention.

FIG. 2 illustrates an exemplary internal configuration of a controller in the first embodiment of the present invention.

FIG. 3 illustrates an example of the positional relationship near a sample and a probe in the first embodiment of the present invention.

FIG. 4 is a flowchart illustrating an operational process in the first embodiment of the present invention.

FIG. 5A illustrates an exemplary reception signal in the first embodiment of the present invention.

FIG. 5B illustrates another exemplary reception signal in the first embodiment of the present invention.

FIG. 6 is a flowchart illustrating a process of determining the state of contact in the first embodiment of the present invention.

FIG. 7A illustrates an example of the positional relationship between the sample and an optical path in the first embodiment of the present invention.

FIG. 7B illustrates another example of the positional relationship between the sample and the optical path in the first embodiment of the present invention.

FIG. 7C illustrates another example of the positional relationship between the sample and the optical path in the first embodiment of the present invention.

FIG. 8A illustrates an example of the positional relationship near the sample in the first embodiment of the present invention.

FIG. 8B illustrates another example of the positional relationship near the sample in the first embodiment of the present invention.

FIG. 8C illustrates another example of the positional relationship near the sample in the first embodiment of the present invention.

FIG. 8D illustrates another example of the positional relationship near the sample in the first embodiment of the present invention.

FIG. 8E illustrates another example of the positional relationship near the sample in the first embodiment of the present invention.

FIG. 9 is a flowchart illustrating a process of determining the state of contact in a second embodiment of the present invention.

FIG. 10 illustrates how to transmit and receive ultrasound beams in the second embodiment of the present invention.

### DESCRIPTION OF EMBODIMENTS

### First Embodiment

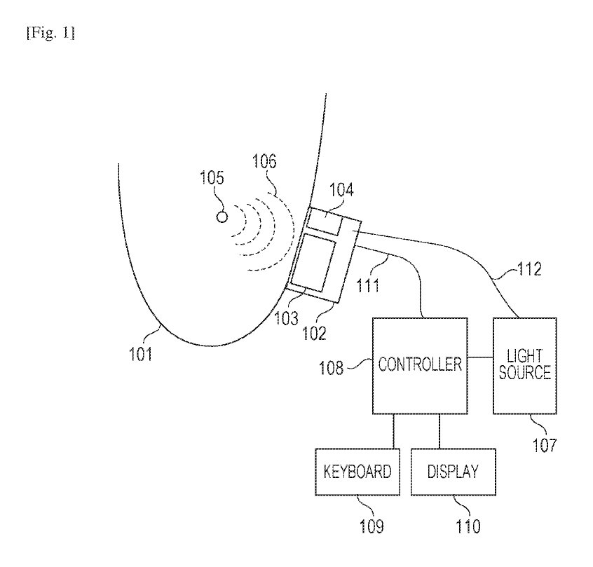

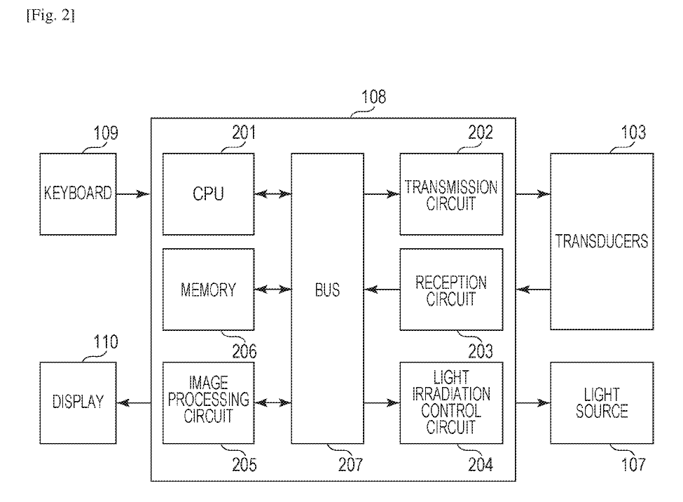

A first embodiment of the present invention will herein be described with reference to FIG. 1 to FIG. 8E. Basic components of the first embodiment will now be described with reference to FIG. 1 and FIG. 2.

The first embodiment includes a light source 107; a light irradiation hole 104, which is a unit of irradiating a sample 101 with light led from the light source 107 through an optical fiber 112; and a probe 102 including multiple transducers 103 that transmit and receive ultrasonic waves to and from the sample 101. The first embodiment also includes an image processing circuit 205, which is an image generation unit that at least generates a photoacoustic image on the basis of a photoacoustic signal of the ultrasonic waves received by the probe 102 for the light radiated from the light irradiation hole 104, which is the light irradiating unit, to the sample 101. In addition, the first embodiment includes a controller 108, which is a determination unit that determines a state of contact between the probe 102 and the sample 101 and whether the sample 101 is on an optical path on the basis of an ultrasonic echo signal of the ultrasonic waves received by the probe 102 for the ultrasonic waves transmitted from the probe 102, prior to the generation of the photoacoustic image. Furthermore, the first embodiment includes a light irradiation control circuit 204, which is a control unit that causes the light irradiation hole 104, which is the light irradiating unit, to irradiate the sample 101 with the light on the basis of results of the determination in the controller 108, which is the determination unit. The controller 108, which is the determination unit, determines the state of contact between the probe 102 and the sample 101 on the basis of information about multiple ultrasonic echo signals received by multiple transducers 103 that are set apart from each other, among the multiple ultrasonic echo signals received by the multiple transducers 103 in response to the ultrasonic waves transmitted from the multiple transducers 103 in the probe 102. With the above configuration, it is possible to perform the determination of whether the sample is on the optical path and the determination of the state of contact between the sample and the probe with a high accuracy in a short time. The reason for this will be described below.

Whether the sample is in contact with the probe is capable of being determined on the basis of the ultrasonic echo signal, which is a signal of reflected ultrasonic waves resulting from reflection of ultrasonic waves transmitted from the probe, as described in PTL1. Specifically, since the reflected ultrasonic waves are generated by air in front of the probe when the probe is not in contact with the sample, the ultrasonic echo signal is received within a very short time immediately after the transmission. Since the propagation path of the ultrasonic waves is very short (the ultrasonic waves are substantially reflected from the surface of the probe), the ultrasonic echo signal is received as the very strong signal with low attenuation. When the ultrasonic echo signal is received as the strong signal within a very short time after the transmission, it is assumed that the probe is not in contact with the sample on the basis of the above characteristics. However, when a layer of air surrounded by the sample exists between the sample and the probe, specifically, for example, when a bubble exists between the sample and the probe, the measurement of the sample may not be affected by the presence of such a non-contact portion depending on the size of the bubble. Also when part of the probe, for example, an end portion of the probe is not in contact with the sample, the measurement of the sample may not be affected depending on the orientation of the probe. Specifically, even when part of the probe is not in contact with the sample, the measurement may not be affected if the probe is oriented to the sample. In other words, it is insufficient to determine whether the sample is irradiated with the light from the light source on the basis of only the determination of whether the sample is in contact with the probe, as in the technology described in PTL1. Also in the determination of the contact, it is desirable to rapidly determine whether the sample is irradiated with the light from the light source in consideration of the entire contact state and the partial contact state.

Accordingly, in embodiments of the present invention, whether the sample is irradiated with the light is determined on the basis of not only information about the state of contact between the sample and the probe but also information about whether the sample is on the optical path. The state of contact between the probe and the sample is determined on the basis of information about multiple ultrasonic echo signals received by multiple transducers that are set apart from each other, among the multiple ultrasonic echo signals received by the multiple transducers in response to the ultrasonic waves transmitted from the multiple transducers in the probe.

With the above method, the state of contact between the sample and the probe is capable of entirely and partially being understood in a short time. Accordingly, it is possible to determine whether the measurement is enabled and to control the light irradiation with a high accuracy in a short time in consideration of partial non-contact, by which the measurement is not affected, for example, partial non-contact between the sample and the probe caused by a bubble or the like, and the orientation of the probe.

The first embodiment will now be described in detail. In addition to the above basic components, various components are included in the first embodiment. The various components will be described in the following description and the basic components may be described again for further understanding of the present invention.

FIG. 1 is a block diagram of a photoacoustic image generation apparatus, which is a sample information acquisition apparatus according to the first embodiment. Referring to FIG. 1, the sample 101 is an object to be measured by the photoacoustic image generation apparatus and is part of the body of a subject. The sample 101 is exemplified by a breast here. The probe 102 includes the transducers 103 used for transmitting and receiving ultrasonic waves to and from the sample and the light irradiation hole 104, which is the unit of irradiating the sample with pulsed light. The transducers 103 are composed of an array of ultrasonic sensor elements, such as lead zirconate titanate (PZT) transducers or capacitive micromachined ultrasonic transducers (CMUTs). The light irradiation hole 104 is an injection hole of the optical fiber and may include optical components, such as a mirror and a diffuser plate.

Reference numeral 105 denotes a portion (light absorber) in the sample, which has high light absorption. The light absorber 105 corresponds to, for example, new blood vessels caused by breast cancer. Upon irradiation of the light absorber 105 with light, such as pulsed light, photoacoustic waves 106 are generated due to a photoacoustic effect. The photoacoustic waves 106 are converted into an electrical signal by the transducers 103 in the probe 102. This electrical signal is referred to as a photoacoustic signal.

The light source 107 generates the pulsed light and is composed of, for example, an yttrium aluminum garnet (YAG) laser or a titanium-sapphire laser. The pulsed laser light source includes a flash lamp as a unit of exciting a laser medium in the light source, which is capable of being electrically controlled from the outside. The pulsed laser light source includes a Q switch, which is capable of being electrically controlled from the outside. Turning on the flash lamp from the outside at a constant frequency, accumulating the excitation energy in the laser medium, and turning on the Q switch cause pulsed light having high energy, which is called giant pulse, to be output.

The controller 108 receives the photoacoustic signal output from the probe 102 and controls the pulsed laser light source 107 and an ultrasonic wave transmission and reception operation by the probe 102. Reference numeral 109 denotes a keyboard used by a user to issue an instruction to start measurement with the photoacoustic image generation apparatus and to input settings of the apparatus. Reference numeral 110 denotes a display used by the user to watch images of the inside of the sample. An appropriate unit, other than the keyboard and the display, may be used as an interface with the user.

Reference numeral 111 denotes a cable to electrically connect the controller 108 to the transducers 103. The optical fiber 112 leads the pulsed light from the pulsed light source 107 to the light irradiation hole 104. The transducers 103 irradiate the sample 101 with the ultrasonic waves on the basis of a signal from the controller 108. In addition, the transducers 103 receive the ultrasonic waves reflected from the sample 101, convert the ultrasonic waves into an electrical signal, and output the electrical signal. This electrical signal is referred to as an ultrasonic echo signal in order to discriminate this electrical signal from the above photoacoustic signal.

Instead of the optical fiber 112, various optical members may be used to connect the light source 107 to the light irradiation hole 104. The optical members include, for example, a mirror that reflects light, a lens that focuses and diffuses light and that changes the shape of the light, a prism that disperses, refracts, and reflects light, an optical fiber through which light is propagated, and a diffuser plate. Any optical member may be used as long as the sample is irradiated with the light that is generated from the light source and that is in a desired shape.

FIG. 2 illustrates an exemplary internal configuration of the controller 108. Referring to FIG. 2, the controller 108 includes a central processing unit (CPU) 201, a transmission circuit 202, a reception circuit 203, the light irradiation control circuit 204, the image processing circuit 205, a memory 206, and a bus 207. The CPU 201 controls the operation of the entire photoacoustic image generation apparatus and is composed of a built-in microcomputer and software. The CPU 201 accepts an instruction from the user with the keyboard 109 and reflects the instruction in the operation of the apparatus. The transmission circuit 202 transmits a high-voltage pulse signal to the transducers 103 and includes a pulser and a transmission memory. The transmission of the ultrasonic waves will now be described. The CPU 201 sets a transmission condition and issues an instruction to start the transmission. The pulse signals are applied to the multiple transducers 103 simultaneously or with a time lag and the ultrasonic waves are generated by the transducers 103. Among the ultrasonic waves generated at this time, the ultrasonic waves transmitted in response to the simultaneous application of the pulse signals to the transducers 103 are referred to as plane waves (plane ultrasound), and the ultrasonic waves transmitted in response to the application of the pulse signals to the transducers 103 with a time lag are referred to as focused ultrasound. The focused ultrasound may be hereinafter referred to as ultrasound beams. In the first embodiment, for example, 32 transducers are used in one transmission and the ultrasound beams, which are the focused ultrasound, are subjected to linear scanning while the elements to be driven are shifted one by one.

The reception circuit 203 receives the ultrasonic echo signal and the photoacoustic signal from the transducers 103 and includes, for example, a preamplifier, an analog-to-digital (A/D) converter, a reception memory, and a field-programmable gate array (FPGA). The ultrasonic echo signal and the photoacoustic signal are amplified by the preamplifier. At this time, the gain of the preamplifier may be varied depending on the input time of the signal to acquire weak signals generated from deeper portions in the sample. The amplified signal is converted into a digital value by the A/D converter and is supplied to the FPGA. Reading and writing data from and into the reception memory and signal processing including noise reduction and phasing addition are performed in the FPGA. The FPGA performs the phasing addition with the phases of the transducers 103 being shifted to generate ultrasonic echo signal data in an arbitrary direction. The ultrasonic echo signal and the photoacoustic signal subjected to the signal processing in the controller 108 are stored in the memory 206 in the controller 108. The pieces of data stored in the memory 206 are referred to as the ultrasonic echo signal data and photoacoustic signal data.

The light irradiation control circuit 204 generates control signals for the flash lamp and the Q-switch in the light source 107. Upon reception of an instruction to radiate the pulsed light from the CPU 201, the image processing circuit 205 generates control pulses for the flash lamp and the Q-switch at a predetermined frequency to cause the light source 107 to generate the pulsed light. Upon reception of an instruction to stop the pulsed light from the CPU 201, the light irradiation control circuit 204 stops the control pulses for the flash lamp and the Q-switch to stop the pulsed light.

The image processing circuit 205, which is the image generation unit, performs image reconfiguration to the photoacoustic signal data to generate an image indicating absorption coefficient distribution of the sample 101 for the pulsed light. The image generation unit (the image processing circuit) also generates image data about the inside of the sample, such as an ultrasound B-mode image, using the ultrasonic echo signal data. The above images are referred to as a photoacoustic image and an ultrasonic echo image. These images are, for example, superimposed with each other and are displayed in the display 110. Only image data in a previous stage about the image to be displayed for the user may be generated in the image processing circuit 205.

The memory 206 temporarily stores the signal data output from the reception circuit 203 and the data output from the image processing circuit 205. The bus 207 is used to connect the circuits in order to receive an instruction from the CPU 201 and exchange data between the circuits.

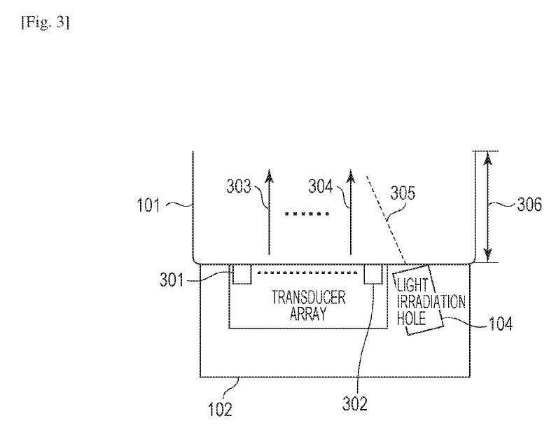

FIG. 3 illustrates an example of the positional relationship near the sample 101 and the probe 102. Referring to FIG. 3, reference numerals 301 and 302 denote transducers in the probe. It is assumed in the first embodiment that the transducers of N-number from 1 (301) to N (302) are sequentially arranged. Reference numerals 303 and 304 denote ultrasound beams transmitted and received to and from the transducers. The controller 108 varies the values of the transmission memory in the transmission circuit 202 and the reception memory in the reception circuit 203 each time the ultrasound beam is transmitted and received to perform the linear scanning of the ultrasound beams. For example, first, the ultrasound beam 303 is generated using the continuous 32 transducers from the transducer 301. The voltage of the transmission circuit 202 is set so that this ultrasound beam has a strength that reaches a depth 306 of the sample 101. Next, the ultrasound beam is generated using the continuous 32 transducers from a transducer next to the transducer 301. The generation of the ultrasound beam is repeated with the transducers being shifted one by one to generate the ultrasound beams. The ultrasound beam 304 is generated using the transducers from the (N-32)-th transducer to the N-th transducer 302 and transmission and reception of the ultrasound beam are performed for the linear scanning. This is repeated across the entire sample 101 to acquire the ultrasonic echo signals in a wide range. Reference numeral 305 denotes an optical path of the pulsed light radiated from the light irradiation hole 104. As illustrated in FIG. 3, the light irradiation hole 104, which is the unit of irradiating the sample 101 with the light, is mounted to the probe 102 and the orientation of the optical path of the light radiated from the light irradiation hole 104 is based on the position of the light irradiation hole 104 and a mounting angle of the light irradiation hole 104 to the main body of the probe.

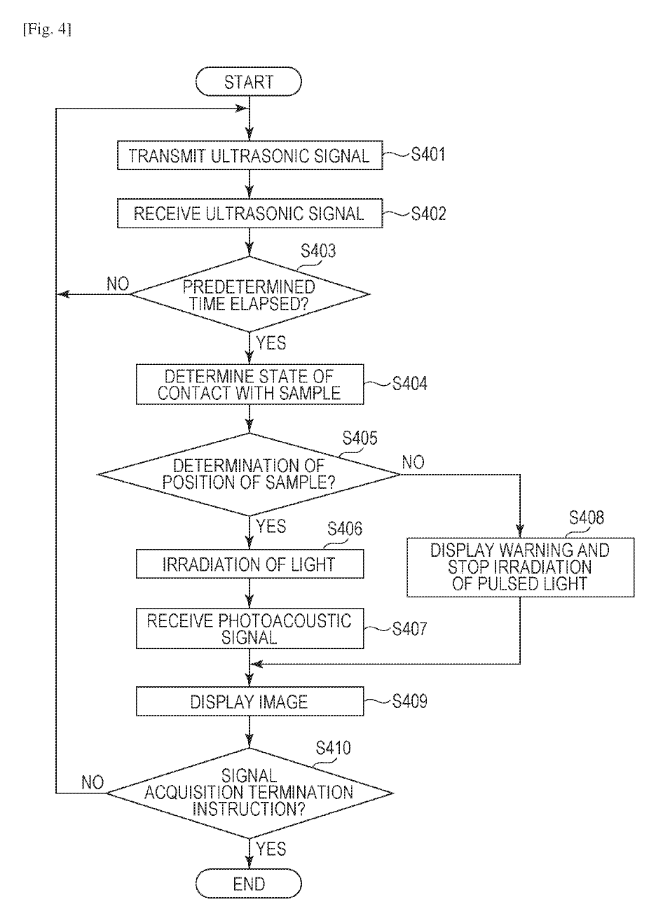

FIG. 4 is a flowchart illustrating an operational process of the photoacoustic image generation apparatus, which is performed by the controller 108.

Referring to FIG. 4, in Step S401, the CPU 201 issues a transmission instruction to the transmission circuit 202 to transmit the ultrasound beam 303.

In Step S402, the ultrasonic echo signal that is generated is received by the probe 102 and the reception circuit 203, is subjected to amplification, digitization, phasing addition, etc., and is stored in the memory 206.

In Step S403, it is determined whether a predetermined time elapsed since the previous pulsed light has been radiated. If the predetermined time elapsed since the previous pulsed light has been radiated (YES in Step S403), the process goes to Step S404. If the predetermined time did not elapse since the previous pulsed light has been radiated (NO in Step S403), the process goes back to Step S401 and the ultrasound beam is transmitted again. The predetermined time in Step S403 is a cycle on which the pulsed light is radiated from the light source 107 and is set to 100 milliseconds in the first embodiment. Since the light source 107 is ready for radiating the next pulsed light when 100 milliseconds elapsed since the previous pulsed light has been radiated in Step S403, the process goes to Step S404.

In Step S404, the CPU 201 analyzes the ultrasonic echo signal data stored in the memory 206 to determine the state of contact between the sample 101 and the probe 102. The CPU 201 also determines whether the sample 101 is on the optical path 305. How to determine the state of contact between the sample 101 and the probe 102 and whether the sample 101 is on the optical path 305 will be described below.

In Step S405, it is determined whether the sample 101 is in good contact with the probe 102 and whether the sample 101 is on the optical path 305. If it is determined that the sample 101 is in good contact with the probe 102 and that the sample 101 is on the optical path 305 (YES in Step S405), the process goes to Step S406. If it is determined that the sample 101 is not on the optical path 305 (NO in Step S405), the process goes to Step S408.

In Step S406, the CPU 201 issues an instruction to radiate the light to the light irradiation control circuit 204 to cause the light source 107 to generate the pulsed light. The pulsed light is radiated from the light irradiation hole 104 to the sample 101.

In Step S407, the reception circuit 203 receives the photoacoustic signal, performs amplification, digitization, noise reduction, etc., to the photoacoustic signal, and stores the photoacoustic signal in the memory 206.

If it is determined that the sample 101 is not in good contact with the probe 102 or that the sample 101 is not on the optical path 305 (NO in Step S405), in Step S408, a warning to the user is displayed and the Q-switch in the light source 107 is stopped to stop the irradiation of the pulsed light. Then, the process goes to Step S409. In the display of the warning, a warning message may be displayed in the display 110 or a display unit such as a light emitting diode (LED), which is provided in addition the display, may be turned on.

In Step S409, the image processing circuit 205 performs image processing including image reconfiguration and scanning conversion to the ultrasonic echo signal and the photoacoustic signal stored in the memory 206 in Step S402 and Step S407, respectively. The ultrasonic echo signal and the photoacoustic signal received from the transducer determined not to be in contact with the sample 101 in Step S404 (the transducer corresponding to a partial non-contact portion between the probe 102 and the sample 101) are not used because such an ultrasonic echo signal and such a photoacoustic signal may cause an artifact. The ultrasonic echo image and the photoacoustic image are generated and are displayed in the display 110. In this case, either of the images may be generated in response to a setting by the user and the generated image may be displayed in the display 110. However, when the photoacoustic signal data is not stored in the memory 206 because it is determined in Step S405 that the sample 101 is not set at a position where the photoacoustic signal is capable of being accurately acquired, only the ultrasonic echo image is displayed.

In Step S410, it is determined whether an instruction to terminate the signal acquisition is issued from the user. If the instruction to terminate the signal acquisition is issued (YES in Step S410), the process in FIG. 4 is terminated. If the instruction to terminate the signal acquisition is not issued (NO in Step S410), the process goes back to Step S401 to repeat the acquisition of the ultrasonic echo signal and the photoacoustic signal.

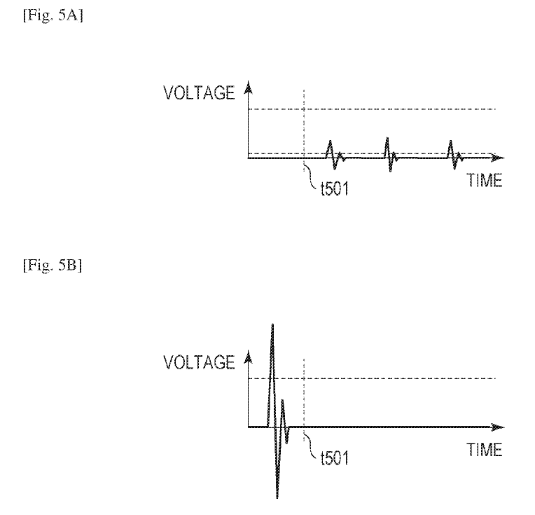

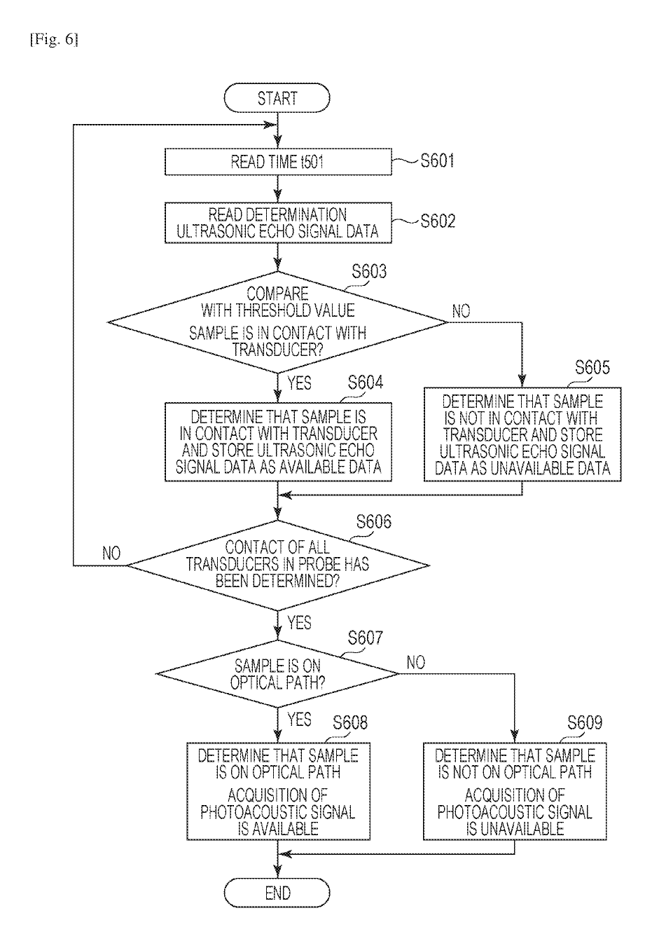

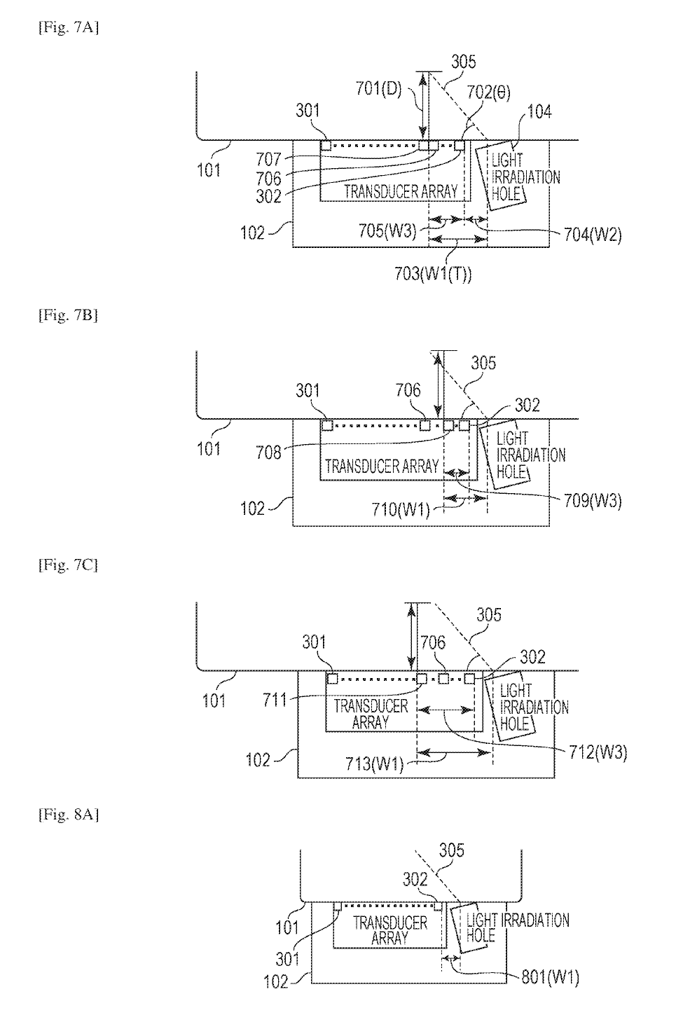

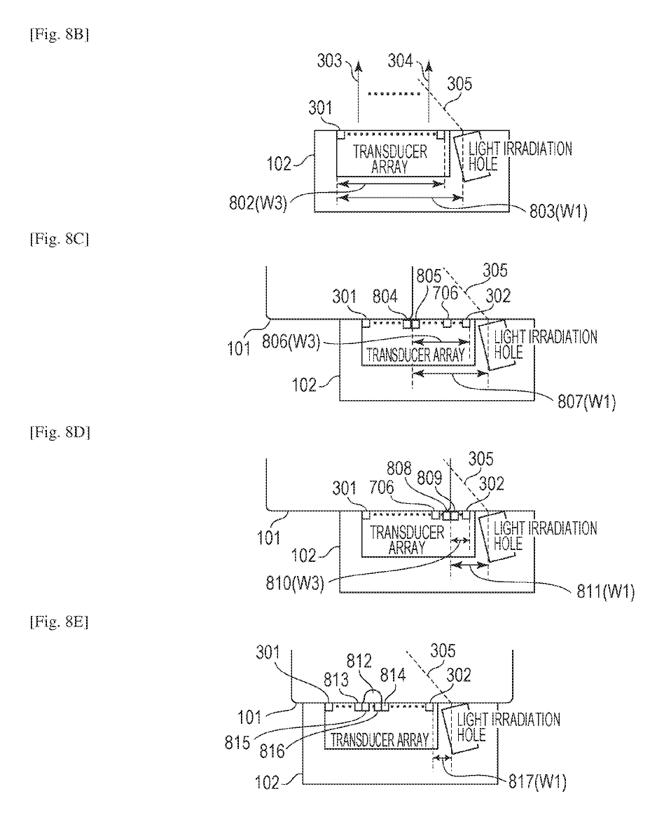

The process of determining the state of contact between the sample 101 and the probe 102 and whether the sample 101 is on the optical path 305 in Step S404 (hereinafter sometimes referred to as a process of determining the position of the sample) will now be described in detail. Although the determination process based on the ultrasonic echo signal data read out from the memory 206 is described in the first embodiment, the determination may be based on the signal (the ultrasonic signal) before the storage in the memory 206. FIG. 5A illustrates the ultrasonic echo signal data when the sample 101 is in contact with the probe 102 and FIG. 5B illustrates the ultrasonic echo signal data when the sample 101 is not in contact with the probe 102. FIG. 6 is a flowchart illustrating the determination process in detail. FIGS. 7A to 7C illustrate examples of the relationship between the sample 101 and the optical path 305. FIGS. 8A to 8E illustrate examples of the positional relationship between the sample 101 and the probe 102.

FIGS. 5A and 5B are graphs illustrating the ultrasonic echo signal data received by a certain transducer 103 when the sample 101 is in contact with the certain transducer 103 and when the sample 101 is not in contact with the certain transducer 103, respectively. Referring to FIGS. 5A and 5B, the vertical axis represents voltage V of the ultrasonic echo signal and the horizontal axis represents time. A time when transmission of the ultrasound beam is started is set to zero. A time t501 will now be described. When the sample 101 is not in contact with the probe 102, most of the ultrasonic waves are reflected between the probe 102 and the air and a large ultrasonic echo signal is received. The time t501 is assumed to be a time when the reception of this large ultrasonic echo signal is completed. The time t501 is calculated in advance on the basis of the configuration of the probe and the speed of sound and is stored in the memory 206. FIG. 5A is a graph in the case in which the sample 101 is in contact with the certain transducer 103. When the sample 101 is in contact with the certain transducer 103, the ultrasonic echo signal has a very low value during a time period before the time t501. FIG. 5B is a graph in the case in which the sample 101 is not in contact with the certain transducer 103. When the sample 101 is not in contact with the certain transducer 103, most of the ultrasonic waves are reflected between the transducer and the air during a time period before the time t501 and a large ultrasonic echo signal is received. Accordingly, the ultrasonic echo signal has a very high value.

FIG. 6 is a flowchart illustrating the process of determining the state of contact between the sample 101 and the probe 102 and whether the sample 101 is on the optical path 305.

Referring to FIG. 6, in Step S601, the time t501 is read out. Then, a time range during which the state of contact between the sample 101 and the probe 102 is determined is set. This time range is referred to as a determination time range. The determination time range is set to a range from zero to the time t501 in the first embodiment. The ultrasonic wave data during the determination time range is referred to as determination ultrasonic echo signal data.

In Step S602, the CPU 201 reads out the determination ultrasonic echo signal data about the first transducer 301 from the memory 206.

In Step S603, the determination ultrasonic echo signal data is compared with a predetermined threshold value. If the number of pieces of data exceeding a threshold value V1 within the determination time range is smaller than M, it is determined that the sample 101 is in contact with this transducer (YES in Step S603). The process goes to Step S604. In Step S604, the ultrasonic echo signal data acquired from this transducer 103 is stored as available data and is stored in the memory 206 with contact information set to one. If the number of pieces of data exceeding the threshold value V1 within the determination time range is M or more, it is determined that the sample 101 is not in contact with this transducer (NO in Step S603). The process goes to Step S605. In Step S605, the ultrasonic echo signal data acquired from this transducer 103 is stored as unavailable data or this unavailable data may be deleted. The ultrasonic echo signal data is stored with the contact information set to zero. Here, M denotes a predetermined natural number and the threshold value V1 is higher than the value of the ultrasonic echo signal from the inside of the sample.

In Step S606, it is determined whether the determination of contact of the transducers is completed to the N-th transducer 302 in the probe 102. If it is determined that the determination of contact of the transducers is completed to the N-th transducer 302 in the probe 102 (YES in Step S606), the process goes to Step S607. If the determination of contact of the transducers is not completed to the N-th transducer 302 in the probe 102 (NO in Step S606), the process goes back to Step S601 to perform the determination of contact of the next transducer in the same manner. The state of contact between the probe 102 and the sample 101 is determined on the basis of data about transducers that are set apart from each other. The state of spacing between transducers may be appropriately set depending on the size of a bubble or the like affecting the measurement, which is set on the basis of the size of the target sample. In the generation of the ultrasonic echo image, image data about the inside of the sample is desirably used without using information about the ultrasonic echo signals the voltage of which exceeds the threshold value V1, among the multiple ultrasonic echo signals received by multiple transducers set apart from each other.

In Step S607, it is determined whether the sample 101 is on the optical path 305. How to determine whether the sample 101 is on the optical path 305 will now be described with reference to FIGS. 7A to 7C. FIGS. 7A to 7C illustrate examples of the relationship between the sample 101 and the optical path 305. FIG. 7A illustrates a case in which the sample 101 intersects with the optical path 305 at a position on the surface of the sample, which corresponds to a depth to be measured of the sample 101. FIG. 7B illustrates a case in which the sample 101 is on the optical path 305. FIG. 7C illustrates a case in which the sample 101 is not on the optical path 305. Reference numeral 701 denotes a depth of the sample 101 (a depth to be measured) and is hereinafter described as D. The depth D is measured in advance and is stored in the memory 206, which is a storage unit. Reference numeral 702 denotes a mounting angle of the light irradiation hole 104 to the probe 102 and is hereinafter described as θ. The mounting angle θ is measured in advance and is stored in the memory 206. Reference numeral 703 denotes a distance from the light irradiation hole 104 along a face of the probe to the sample 101 and is hereinafter described as a distance W1. Reference numeral 704 denotes a distance from the light irradiation hole 104 along the face of the probe to the nearest transducer 302 and is hereinafter described as W2. The distance W2 is measured in advance and is stored in the memory 206. Reference numeral 705 denotes a distance from the transducer 302 along the face of the probe to the sample 101 and is hereinafter described as W3. How to calculate the distance W3 will now be described. First, the number of transducers between the transducer 302 and a transducer having the contact information of one is counted. The distance W3 is calculated by multiplying the number of the transducers by a distance between the transducers. The distance between the transducers is measured in advance and is stored in the memory 206. The distance W1 is calculated by adding the distance W3 to the distance W2. Transducers 706 and 707 in FIG. 7A represent transducers on the boundary between the transducer that is in contact with the sample 101 and the transducer that is not in contact with the sample 101. The transducer 706 represents the transducer on the boundary at a side where the transducer is not in contact with the sample 101. The transducer 707 represents the transducer on the boundary at a side where the transducer is in contact with the sample 101.

W1 (703) in FIG. 7A is referred to as a light irradiation contact distance threshold value, which is hereinafter described as T. The light irradiation contact distance threshold value T is calculated according to Equation (1) on the basis of FIG. 7A. The light irradiation contact distance threshold value T is calculated in advance and is stored in the memory 206. This means that the transducer number of the transducer 706 or 707 is stored in the memory 206

$$\begin{matrix}[ Math . 1 ] & \\ T = \frac{D}{tan θ} & ( 1 )\end{matrix}$$

If the distance W1, which is the distance between the sample 101 and the light irradiation hole 104, is shorter than the light irradiation contact distance threshold value T, this means that the distance between the sample 101 and the light irradiation hole 104 is shorter than that when the sample 101 intersects with the optical path 305 at an innermost portion of the sample 101. In other words, it is determined that the sample 101 is on the optical path 305. In contrast, if the distance W1, which is the distance between the sample 101 and the light irradiation hole 104, is longer than the light irradiation contact distance threshold value T, this means that the distance between the sample 101 and the light irradiation hole 104 is longer than that when the sample 101 intersects with the optical path 305 at the innermost portion of the sample 101. In other words, it is determined that the sample 101 is not on the optical path 305. Accordingly, the comparison between the distance W1 with the light irradiation contact distance threshold value T enables the determination of whether the sample 101 is on the optical path 305.

The case in which the sample 101 is on the optical path 305 will now be described with reference to FIG. 7B. Referring to FIG. 7B, a transducer 708 is at the side where the transducer is not in contact with the sample 101 on the boundary between the transducer that is in contact with the sample 101 and the transducer that is not in contact with the sample 101, between the sample 101 and the probe 102. The transducer 708 is between the transducer 302 and the transducer 706 described above on the boundary when the surface of the sample corresponding to the depth to be measured of the sample intersects with the optical path. This means that the distance W3 (709) in FIG. 7B is shorter than the distance W3 (705) in FIG. 7A. Relational expression (2) is established between the distance W1 (710) in FIG. 7B and the light irradiation contact distance threshold value T.

W1<T (2)

In this case, it is determined that the sample 101 is on the optical path 305 (YES in Step S607). In Step S608, it is determined that the sample 101 is on the optical path 305 and that the acquisition of the photoacoustic signal is available. Then, the process illustrated in FIG. 6 is terminated.

The case in which the sample 101 is not on the optical path 305 will now be described with reference to FIG. 7C. Referring to FIG. 7C, a transducer 711 at the side where the transducer is not in contact with the sample 101, among the transducers on the boundary between the transducer that is in contact with the sample 101 and the transducer that is not in contact with the sample 101, is between the transducer 301 and the transducer 706 described above on the boundary when the surface of the sample corresponding to the depth to be measured of the sample intersects with the optical path. Since the distance W3 (712) in FIG. 7C is longer than the distance W3 (705) in FIG. 7A, Relational expression (3) is established between the distance W1 (713) in FIG. 7C and the light irradiation contact distance threshold value T.

W1>T (3)

In this case, it is determined that the sample 101 is not on the optical path 305 (NO in Step S607). In Step S609, it is determined that the sample 101 is not on the optical path 305 and that the acquisition of the photoacoustic signal is unavailable. Then, the process illustrated in FIG. 6 is terminated. As described above, whether the sample is on the optical path is determined on the basis of the relationship between the distance W1 and the light irradiation contact distance threshold value T, that is, information about the mounting angle of the light irradiation hole, which is the unit of irradiating the sample with the light, to the probe. The information is stored in the memory, which is the storage unit. The transducer 708 and the transducer 711, which am the transducers on the boundary between the transducer that is in contact with the sample and the transducer that is not in contact with the sample, are capable of being determined by sequentially confirming only the ultrasonic echo signals of the transducers between the transducer that is in contact with the sample and the transducer that is not in contact with the sample, among the ultrasonic echo signals received by transducers set apart from each other.

How the determination of the state of contact between the sample 101 and the probe 102 and whether the sample 101 is on the optical path 305 is varied depending on the positional relationship between the sample 101 and the probe 102 will now be described with reference to FIGS. 8A to 8E.

FIG. 8A illustrates an exemplary case in which the sample 101 is in contact with the entire surface of the probe 102 and the light is incident on the sample 101. FIG. 8B illustrates an exemplary case in which the sample 101 is not positioned in front of the probe 102. FIG. 8C illustrates an exemplary case in which, although the sample 101 is positioned in front of the probe 102, the position of the sample 101 is shifted from the probe 102 and the sample 101 is not irradiated with the light. FIG. 8D illustrates an exemplary case in which the sample 101 is positioned in front of the probe 102 and the light is incident on the sample 101 although part of the sample 101 is not in contact with the probe 102. FIG. 8E illustrates an exemplary case in which a bubble 812 exists between the sample 101 and the probe 102.

In the exemplary case in FIG. 8A, no air exists between the sample 101 and the probe 102 and the sample 101 is in contact with the probe 102. Accordingly, most of the ultrasound beams are propagated into the inside of the sample and are gradually attenuated. Consequently, the number of pieces of data in which the determination ultrasonic echo signal exceeds the threshold value V1 within the determination time range is smaller than or equal to M for all the transducers set apart from each other. As a result, it is determined in Step S603 that the entire reception face of the probe 102 is in contact with the sample 101. In addition, since the transducer 302 is in contact with the sample 101 in Step S607, the distance W3 in FIG. 8A is equal to zero. Accordingly, since the relationship W1<T is established between the distance W1 (801) in FIG. 8A and the light irradiation contact distance threshold value T, it is determined that the sample 101 is on the optical path 305 and the acquisition of the photoacoustic signal is available.

In the exemplary case in FIG. 8B, the sample 101 is not in contact with the probe 102 because the sample 101 is not positioned in front of the probe 102. Accordingly, most of the ultrasound beams from the ultrasound beam 303 to the ultrasound beam 304 are reflected from the boundary between the surface of the probe and the air. Consequently, as illustrated in FIG. 5B, the ultrasonic echo signal of a high voltage appears near the time t501 immediately after the start of the reception. As a result, it is determined in Step S603 that all the transducers set apart from each other are not in contact with the sample 101 and that the probe 102 is not in contact with the sample 101. Since all the transducers are not in contact with the sample 101 in Step S607, the distance W3 (802) in FIG. 8B corresponds to the entire width of the array of transducers in the probe 102 and has a maximum value in the first embodiment. Accordingly, since the distance W1 (803) in FIG. 8B also has a maximum value in the first embodiment, the relationship W1>T is established between the distance W1 (803) in FIG. 8B and the light irradiation contact distance threshold value T. Consequently, it is determined that the sample 101 is not on the optical path 305 and the acquisition of the photoacoustic signal is unavailable.

In the exemplary case in FIG. 8C, the sample 101 is partially in contact with the probe 102. Accordingly, among the transducers set apart from each other from the transducer 301 to a transducer 804 in the probe 102, the number of pieces of data in which the determination ultrasonic echo signal exceeds the threshold value V1 within the determination time range is smaller than M for each of the transducers set apart from each other. As a result, it is determined in Step S603 that the transducers from the transducer 301 to the transducer 804 are in contact with the sample 101. In contrast, among the transducers set apart from each other from a transducer 805 to the transducer 302 in the probe 102, the number of pieces of data in which the determination ultrasonic echo signal exceeds the threshold value V1 within the determination time range is greater than or equal to M for each of the transducers set apart from each other. As a result, it is determined in Step S603 that the transducers from the transducer 805 to the transducer 302 in the probe 102 are not in contact with the sample 101 and that the probe 102 is not in contact with the sample 101 in a portion from the transducer 805 to the transducer 302. Since the transducer 805 at the side where the transducer is not in contact with the sample 101 on the boundary between the transducer that is in contact with the sample 101 and the transducer that is not in contact with the sample 101 is between the transducer 301 and the transducer 706 described above and the transducer 302 is also not in contact with the sample 101, it is determined that the state of contact between the probe 102 and the sample 101 is not good. Since the transducer 805 at the side where the transducer is not in contact with the sample 101 on the boundary between the transducer that is in contact with the sample 101 and the transducer that is not in contact with the sample 101 is between the transducer 301 and the transducer 706 described above, the distance W3 (806) in FIG. 8C is longer than the distance W3 (705) in FIG. 7A in Step S607. Accordingly, since the relationship W1>T is established between the distance W1 (807) in FIG. 8C and the light irradiation contact distance threshold value T, it is determined that the sample 101 is not on the optical path 305 and the acquisition of the photoacoustic signal is unavailable.

In the exemplary case in FIG. 8D, the sample 101 is partially in contact with the probe 102. Accordingly, among the transducers set apart from each other from the transducer 301 to a transducer 808 in the probe 102, the number of pieces of data in which the determination ultrasonic echo signal exceeds the threshold value V1 within the determination time range is smaller than M for each of the transducers set apart from each other. As a result, it is determined in Step S603 that the transducers from the transducer 301 to the transducer 808 are in contact with the sample 101. In contrast, among the transducers set apart from each other from a transducer 809 to the transducer 302 in the probe 102, the number of pieces of data in which the determination ultrasonic echo signal exceeds the threshold value V1 within the determination time range is greater than or equal to M for each of the transducers set apart from each other. As a result, it is determined in Step S603 that the probe 102 is not in contact with the sample 101 in a portion from the transducer 809 to the transducer 302. However, since the transducer 809 at the side where the transducer is not in contact with the sample 101 on the boundary between the transducer that is in contact with the sample 101 and the transducer that is not in contact with the sample 101 is between the transducer 706 described above and the transducer 302, it is determined that the measurement of the sample is not affected in the non-contact portion (the non-contact portion is sufficiently small) and that the state of contact between the sample 101 and the probe 102 is good. In addition, since the transducer 809 at the side where the transducer is not in contact with the sample 101 on the boundary between the transducer that is in contact with the sample 101 and the transducer that is not in contact with the sample 101 is between the transducer 706 described above and the transducer 302, the distance W3 (810) in FIG. 8D is shorter than the distance W3 (705) in FIG. 7A in Step S607. Accordingly, since the relationship W1<T is established between the distance W1 (811) in FIG. 8D and the light irradiation contact distance threshold value T, it is determined that the sample 101 is on the optical path 305 and the acquisition of the photoacoustic signal is available.

In the exemplary case in FIG. 8E, the sample 101 is in contact with the probe 102 except for a portion corresponding to the bubble 812. Accordingly, among the transducers set apart from each other from the transducer 301 to a transducer 813 and from a transducer 814 to the transducer 302 in the probe 102, the number of pieces of data in which the determination ultrasonic echo signal exceeds the threshold value V1 within the determination time range is smaller than M for each of the transducers set apart from each other. As a result, it is determined in Step S603 that the transducers from the transducer 301 to the transducer 813 and from the transducer 814 to the transducer 302 are in contact with the sample 101. In contrast, among the transducers set apart from each other from a transducer 815 to a transducer 816 in the probe 102, the number of pieces of data in which the determination ultrasonic echo signal exceeds the threshold value V1 within the determination time range is greater than or equal to M for each of the transducers set apart from each other. As a result, it is determined in Step S603 that the probe 102 is not in contact with the sample 101 in a portion from the transducer 815 to the transducer 816. However, since it is determined that the transducer 302 is in contact with the sample 101, it is determined that the non-contact portion is surrounded by the sample 101 (is the bubble or the like) and that the state of contact between the probe 102 and the sample 101 is good. As described above, it is determined that the state of contact is good when the light irradiation has no trouble even if the non-contact portion of a certain size exists. It may be determined that, even when the non-contact portion is surrounded by the sample (for example, a bubble), the measurement is disabled depending on the size of the non-contact portion. In such a case, the settings may be appropriately varied depending on the size or the like of the object to be measured. In addition, since it is determined in Step S607 that the transducer 302 is in contact with the sample 101, the distance W3 in FIG. 8E is equal to zero. Accordingly, since the relationship W1<T is established between the distance W1 (817) in FIG. 8E and the light irradiation contact distance threshold value T, it is determined that the sample 101 is on the optical path 305 and the acquisition of the photoacoustic signal is available.

As described above, the determination of the state of contact between the sample 101 and the probe 102 using the multiple pieces of determination ultrasonic echo signal data received by multiple transducers set apart from each other allows the partial state of contact and the entire state of contact to be determined even when it is difficult to make the sample in contact with the entire probe, for example, in the case of the periphery of a breast or in the case of an arm that is slim. Accordingly, it is possible to determine whether the state of contact between the sample and the probe is good. Specifically, even when the bubble 812 or the like exists between the sample 101 and the probe 102, the determination of the state of contact of the probe 102 allows the transducers in the bubble portion to be determined not to be in contact with the sample and allows the transducers in portions excluding the bubble to be determined to be in contact with the sample. In addition, it is also possible to determine whether the sample 101 is on the optical path 305 and it is possible to control the light irradiation to the sample on the basis of the results of the determination. In the generation of the ultrasonic echo image and the photoacoustic image, no use of the ultrasonic echo signal data and the photoacoustic signal data about the transducers that are not in contact with the sample allows an image with a small artifact to be provided.

In the first embodiment, the reception circuit 203 writes the ultrasonic echo signal data into the memory 206 and the CPU 201 reads out the ultrasonic echo signal data stored in the memory 206 to determine the position of the sample. However, the timing of the determination is not limited to this. For example, the reception circuit 203 may determine the position of the sample in the writing of the ultrasonic echo signal data into the memory 206 in order to reduce the time to read and write data from and into the memory.

Although the probe including a one-dimensional array is described in the first embodiment for simplicity, the state of contact between the sample and the probe is capable of being determined for a probe including a two-dimensional array and whether the sample is on the optical path is capable of being determined on the basis of the information about the contact and the non-contact.

Although the Q-switch in the light source is stopped to stop the irradiation of the sample with the pulsed light when it is determined that the acquisition of the photoacoustic signal is unavailable in the first embodiment, the method of irradiating the sample with the pulsed light is not limited to this. For example, a shutter may be provided outside the light source and the shutter may be closed to stop the irradiation of the sample with the pulsed light.

Although the example in which one light irradiation hole is provided at a side of the transducers is described in the first embodiment, the position of the light irradiation hole and the number of the light irradiation holes in the photoacoustic apparatus are not limited to the above ones. For example, the light irradiation holes may be provided on both sides of the probe. In this case, the state of contact between the sample and the probe is capable of being determined in the same manner as in the first embodiment. In addition, whether the sample is on the optical path is capable of being determined using the states of contact of the transducers on both sides of the probe.

As described above, it is possible to determine the partial contact relationship and the entire contact relationship between the sample and the probe and the positional relationship with the optical path using the ultrasound beams in the photoacoustic apparatus in the first embodiment to determine in advance whether the photoacoustic apparatus is in a state in which the photoacoustic signal is capable of being accurately acquired. As a result, the accuracy of the acquired photoacoustic signal may be increased to improve the diagnostic accuracy. In addition, non-irradiation of the sample with the pulsed light in the situation in which the photoacoustic signal is not capable of being accurately acquired allows the life of the apparatus to be lengthened. Such advantages are similarly achieved also in a case in which a light irradiation hole and transducers are provided in a hand-held probe and also in a case of a bed-type photoacoustic apparatus. Furthermore, the use of information about the signals received by part of the transducers set apart from each other for the determination of the state of contact between the sample and the probe allows the time to acquire the photoacoustic signal to be reduced, compared with a case in which all the transducers are used. As a result, it is possible to suppress an occurrence of variation in the relative position between the sample 101 and the probe 102, which is caused by a motion of the sample or the like, to generate an excellent diagnostic image.

### Second Embodiment

A second embodiment of the present invention will now be described. The second embodiment differs from the first embodiment in that, in the determination of the position of the sample, the determination is based on the pieces of ultrasonic echo signal data that are acquired by newly transmitting and receiving the ultrasonic waves, instead of the reading out of the pieces of ultrasonic echo signal data that have been received for analysis. In other words, transmission and reception of determination ultrasound beams are performed prior to the irradiation of the sample with the light for receiving the photoacoustic waves.

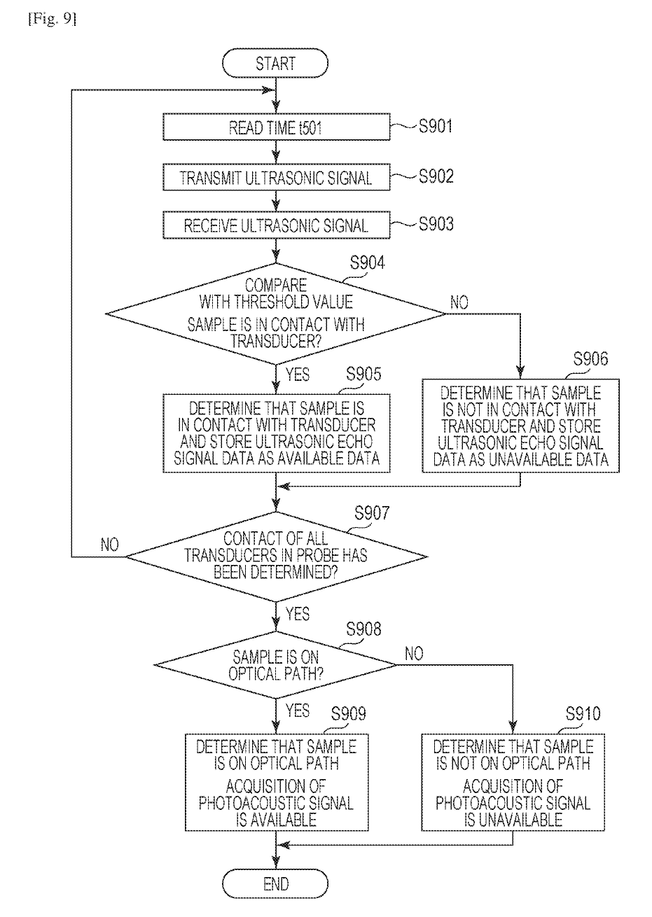

Since the block configuration and the operational flow in the second embodiment are the same as those in the first embodiment, a description of the block configuration and the operational flow in the second embodiment is omitted herein. FIG. 9 is a flowchart illustrating a process of determining the state of contact between the sample 101 and the probe 102 and whether the sample 101 is on the optical path 305 in the second embodiment.

Referring to FIG. 9, in Step S901, the CPU 201 reads out the time t501, as in the first embodiment.

In Step S902, the CPU 201 issues an instruction to the transmission circuit 202 to transmit the ultrasound beams to the sample 101. Since it is sufficient for the ultrasound beams to have a strength that reaches the surface of the sample, making the voltage of the transmission circuit 202 low allows the Lime to transmit and receive the ultrasound beams to be reduced.

In Step S903, the ultrasonic echo signal of the ultrasound beams is received with the reception circuit 203 for digitization. Transmission and reception of the ultrasound beams will be described below.

In Step S904, the ultrasonic echo signal data within the determination time range calculated in Step S901, among the received pieces of the ultrasonic echo signal data, is compared with the predetermined threshold value V1, as in the first embodiment.

In Steps S905 to S910, processing similar to that in Step S604 to S609 in the first embodiment is performed to determine whether the sample 101 is on the optical path 305.

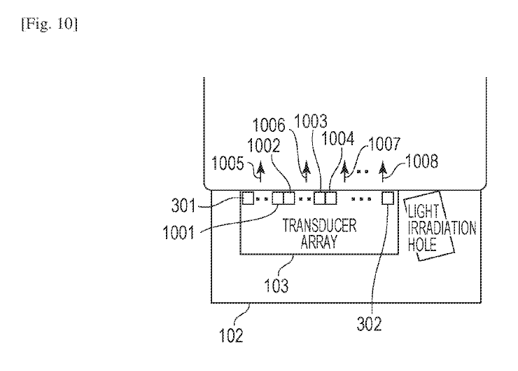

FIG. 10 illustrates how to transmit and receive the ultrasound beams in the determination of the state of contact between the sample and the probe in the second embodiment. Referring to FIG. 10, reference numerals 1001, 1002, 1003, and 1004 denote multiple transducers. Reference numerals 1005, 1006, 1007, and 1008 denote ultrasound beams.

In the second embodiment, transducers set apart from each other, among the transducers from the transducer 301 to the transducer 1001, are used in the first transmission and reception. The ultrasound beam generated at this time is the ultrasound beam 1005. Transducers set apart from each other, among the transducers from the transducer 1002 next to the transducer 1001 to the transducer 1003, are used in the next transmission and reception. The ultrasound beam generated at this time is the ultrasound beam 1006. This is repeated to perform only one transmission and reception for the respective transducers set apart from each other, among all the transducers from the transducer 1004 to the transducer 302, and determine the state of contact, thereby speeding up the determination. The ultrasound beams from the ultrasound beam 1007 to the ultrasound beam 1008 are generated by the transducers between the transducer 1004 and the transducer 302. However, the above transmitting and receiving method is not limitedly used. For example, transmission and reception may be performed at a time using the respective transducers set apart from each other in all the transducers to determine the state of contact. Alternatively, transmission and reception may be performed multiple times for the respective transducers set apart from each other to determine the state of contact or transmission and reception may be performed multiple times for only part of the transducers to determine the state of contact.

In the second embodiment, the determination is performed using the ultrasonic echo signal data acquired by radiating the ultrasound beams immediately before the sample is irradiated with the pulsed light. Accordingly, it takes a time to transmit and receive the ultrasonic waves in Step S401 to Step S403 and to acquire the photoacoustic signal in Step S407. Consequently, even when the relative position between the sample 101 and the probe 102 is varied, it is possible to accurately determine the positional relationship between the sample 101 and the probe 102.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2014-249436, filed Dec. 9, 2014, which is hereby incorporated by reference herein in its entirety.

### REFERENCE SIGNS LIST

101 sample
102 probe
103 transducer
104 light irradiation hole
107 light source
201 CPU
204 light irradiation control circuit
205 image processing circuit

1. A sample information acquisition apparatus comprising:
a light source;
an irradiation member optically connected to the light source configured to irradiate a sample with light;
a probe including a plurality of transducers that transmit and receive ultrasonic waves to the sample;
an image generation unit configured to at least generate a photoacoustic image on the basis of a photoacoustic signal of the ultrasonic waves received by the probe for the light radiated from the irradiation member to the sample;
a determination unit configured to determine a state of acoustic contact between the probe and the sample and whether the sample is on an optical path on the basis of an ultrasonic echo signal of the ultrasonic waves received by the probe for the ultrasonic waves transmitted from the probe, prior to the generation of the photoacoustic image; and
a control unit configured to cause the irradiation member to irradiate the sample with the light on the basis of a result of the determination in the determination unit,
wherein the determination unit determines the state of acoustic contact between the probe and the sample on the basis of information about a plurality of ultrasonic echo signals received by a plurality of transducers that are set apart from each other, among the plurality of ultrasonic echo signals received by the plurality of transducers in response to the ultrasonic waves transmitted from the plurality of transducers in the probe.

2. The sample information acquisition apparatus according to claim 1, wherein the determination unit determines the state of acoustic contact between the probe and the sample on the basis of whether voltage of the ultrasonic echo signal exceeds a predetermined threshold value.

3. The sample information acquisition apparatus according to claim 2, wherein the image generation unit also generates image data about inside portions of the sample using the ultrasonic echo signal.

4. The sample information acquisition apparatus according to claim 3, wherein the image generation unit generates the image data about the inside portions of the sample without using information about the ultrasonic echo signal the voltage of which exceeds the predetermined threshold value, among the plurality of ultrasonic echo signals received by the plurality of transducers set apart from each other.

5. The sample information acquisition apparatus according to claim 1, wherein the ultrasonic waves transmitted from the probe are plane waves.

6. The sample information acquisition apparatus according to claim 1, wherein the ultrasonic waves transmitted from the probe are focused ultrasound.

7. The sample information acquisition apparatus according to claim 6, wherein the probe performs linear scanning of the focused ultrasound.

8. The sample information acquisition apparatus according to claim 1, wherein the unit of irradiating the sample with the light is mounted to the probe.

9. The sample information acquisition apparatus according to claim 8, further comprising:
a storage unit configured to store a mounting angle of the unit of irradiating the sample with the light to the probe.

10. The sample information acquisition apparatus according to claim 9, wherein the determination unit determines whether the sample is on the optical path on the basis of information about the mounting angle of the unit of irradiating the sample with the light to the probe, which is stored in the storage unit.

11. The sample information acquisition apparatus according to claim 1, further comprising a housing configured to be held by a hand of an operator;
wherein the light source and the plurality of transducers are installed in the housing in built-in manner.
